(19) United States
(12) Patent Application Publication
(10) Pub. No.: US 2026/0056189 A1
(43) Pub. Date: Feb. 26, 2026

(54) CENTRIFUGAL IMMUNOASSAY DEVICE FOR MULTIPLE DIAGNOSTICS

(71) Applicants: Soulbrain Holdings Co.,Ltd., Seongnam-si (KR); igenetech Inc., Seoul (KR)

(72) Inventors: Myoung Soon KIM, Seongnam-si (KR); Mee Hyun JEOUNG, Seongnam-si (KR); Ji Soo LEE, Seongnam-si (KR); Hyung Mo KIM, Seongnam-si (KR)

(21) Appl. No.: 18/899,817

(22) Filed: Sep. 27, 2024

(30) Foreign Application Priority Data
Aug. 22, 2024 (KR) 10-2024-0112723

(51) Int. Cl. G01N 33/53; B04B 5/04; B04B 13/00; G01N 21/03; G01N 21/31

(52) U.S. Cl. CPC G01N 33/5302; B04B 5/0414; B04B 13/00; G01N 21/03; G01N 21/31

(57) ABSTRACT

An embodiment relates to a centrifugal immunoassay device for multiple diagnoses for detecting an object to be detected from a sample through centrifugation, the device including: a centrifugal cartridge having a plurality of wells in which the sample is received; a rotating part rotating in a state of mounting the centrifugal cartridge thereon; an absorbance optical system coupled to the centrifugal cartridge to measure the absorbance of the object to be detected received in a detection well among the plurality of wells of the centrifugal cartridge; a syringe adapted to be inserted into the centrifugal cartridge, suck the object to be detected, and discharge the sucked object to the detection well; and a housing for surrounding the centrifugal cartridge, the rotating part, the absorbance optical system, and the syringe.

### CROSS REFERENCE TO RELATED APPLICATION OF THE DISCLOSURE

The present application claims the benefit of Korean Patent Application No. 10-2024-0112723 filed in the Korean Intellectual Property Office on Aug. 22, 2024, the entire contents of which are incorporated herein by reference.

### BACKGROUND OF THE DISCLOSURE

### Field of the Disclosure

The present disclosure relates to a device, more specifically to a centrifugal immunoassay device for multiple diagnoses.

### Background of the Related Art

A cartridge in a conventional immunoassay device is configured to have a structure of a rotary type disk capable of allowing automatic measurements, and further, the cartridge is designed to treat a specimen in its chambers. In this case, however, the conventional immunoassay device depends upon the rotating disk so that it needs fine paths, and further, joining an upper plate and a lower plate is complicated, thereby causing difficulties in mass production and management thereof. Besides, a plurality of parts should be needed in making the cartridge, thereby raising a manufacturing cost of the cartridge, and additionally, it is hard to inject the specimen into the cartridge. Further, the conventional immunoassay device is provided with an absorbance optical system having a light source with a single wavelength, thereby making it possible to achieve only a single diagnosis.

Furthermore, the conventional immunoassay device has a centrifuge and a transmission type detector provided separately from each other, which additionally requires a step of moving an object to be detected from the centrifuge to the transmission type detector. As a result, undesirably, a detection time and cost may increase.

To solve such problems occurring in the conventional immunoassay device, there is a need to develop a new centrifugal immunoassay device that is provided with a cartridge simple in configuration and having a plurality of wells capable of allowing centrifugation and transmission type detection so that the centrifugation and the transmission type detection are performed through the single cartridge, thereby improving a user's conveniences. Further, there is a need to develop a new centrifugal immunoassay device that is capable of performing multiple diagnoses through an absorbance optical system having compact multiple color optical components.

### PRIOR ART DOCUMENT

[Patent document]
Korean Patent Application Laid-open No. 10-2024-0018037 (Feb. 13, 2024)

### SUMMARY OF THE DISCLOSURE

Accordingly, the present disclosure has been made in view of the above-mentioned problems occurring in the related art, and it is an object of the present disclosure to provide a centrifugal immunoassay device for multiple diagnoses that is capable of having a plurality of wells formed on a single cartridge to allow centrifugation and transmission type detection to be performed in the single cartridge and capable of having an optical system adapted to measure a plurality of wavelengths, thereby making it possible to perform the multiple diagnoses.

To accomplish the above-mentioned object, according to the present disclosure, there is provided a centrifugal immunoassay device for multiple diagnoses for detecting an object to be detected from a sample through centrifugation, comprising: a centrifugal cartridge having a plurality of wells in which the sample is received; a rotating part rotating in a state of mounting the centrifugal cartridge thereon; an absorbance optical system coupled to the centrifugal cartridge to measure the absorbance of the object to be detected received in a detection well among the plurality of wells of the centrifugal cartridge; a syringe adapted to be inserted into the centrifugal cartridge, suck the object to be detected, and discharge the sucked object to the detection well; and a housing for surrounding the centrifugal cartridge, the rotating part, the absorbance optical system, and the syringe, wherein the absorbance optical system comprises: a plurality of light emission parts for emitting light having given wavelengths in different ranges to the object to be detected received in the detection well; a light detection part for measuring the light passing through the object to be detected received in the detection well to calculate a detection value of the light; and a light selection part for selecting the light having the given wavelength in any one range emitted from one of the plurality of light emission parts to transmit the selected light to the objected to be detected received in the detection well.

According to the present disclosure, desirably, the sample may be blood and the detection object may be plasma.

According to the present disclosure, desirably, the detection well may have the shape of a cuvette capable of allowing transmission type detection.

According to the present disclosure, desirably, the centrifugal cartridge may include: a sample well for receiving the sample therein; a centrifugal well for receiving the sample moving thereto from the sample well through rotations; a sample-moving path formed by connecting the sample well and the centrifugal well to each other; and the detection well for receiving the object to be detected from the syringe.

According to the present disclosure, desirably, the syringe may have a tip member located on the bottom thereof and the tip member may pass through a top of the centrifugal cartridge, suck the detection object received in the centrifugal well, and then discharge the sucked detection object to the detection well.

According to the present disclosure, desirably, the centrifugal well may have a stepped portion formed on a lower portion thereof to suck the detection object separated from the sample and the tip member may be inserted into an upper portion of the stepped portion to suck the detection object.

According to the present disclosure, desirably, the plurality of light emission parts may be arranged in the form of a circle, and the light selection part may include a driving motor for rotating the plurality of light emission parts arranged in the form of the circle and a position sensor for detecting the positions of the plurality of light emission parts.

According to the present disclosure, desirably, each light emission part may include: a color LED for emitting the light having the given wavelength; an LED lens for focusing the light emitted from the color LED; and a bandpass filter for passing the light having the given wavelength therethrough.

According to the present disclosure, desirably, the housing may include at least any one of a printer, a touch screen, a progress indicator, a power switch, and a door.

According to the present disclosure, desirably, the touch screen may receive commands for performing operations of the centrifugal immunoassay device for multiple diagnoses from a user.

### BRIEF DESCRIPTION OF THE DRAWINGS

The above and other objects, features and advantages of the present disclosure will be apparent from the following detailed description of the preferred embodiments of the disclosure in conjunction with the accompanying drawings, in which:

FIG. 1 is a perspective view showing a centrifugal immunoassay device according to the present disclosure, in which a housing is removed;

FIG. 2 is a perspective view showing a centrifugal cartridge of the centrifugal immunoassay device according to the present disclosure;

FIG. 3 is a sectional view showing the centrifugal cartridge according to the present disclosure;

FIG. 4 is a perspective view showing an absorbance optical system of the centrifugal immunoassay device according to the present disclosure;

FIG. 5 is a schematic view showing the absorbance optical system of the centrifugal immunoassay device according to the present disclosure; and

FIGS. 6 and 7 are perspective views showing the centrifugal immunoassay device according to the present disclosure.

### DETAILED DESCRIPTION OF THE PREFERRED EMBODIMENTS

Hereinafter, an embodiment of the present disclosure will be explained in detail with reference to the attached drawings, and further, the embodiment may be embodied in different forms and should not be construed as limited to the embodiments set forth herein but may be modified and variously implemented by those skilled in the art. If it is determined that the detailed explanation on the well-known technology related to the present disclosure makes the scope of the present disclosure not clear, the explanation will be avoided for the brevity of the description, and in the drawings, the corresponding parts in the embodiment of the present disclosure are indicated by corresponding reference numerals.

When it is said that one element is described as being “connected” or “coupled” to the other element, one element may be directly connected or coupled to the other element, but it should be understood that another element may be present between the two elements. In contrast, when it is said that one element is described as being “directly connected” or “directly coupled” to the other element, it should be understood that another element is not present between the two elements.

Terms used in this application are used to only describe specific exemplary embodiments and are not intended to restrict the present disclosure. An expression referencing a singular value additionally refers to a corresponding expression of the plural number, unless explicitly limited otherwise by the context. In this application, terms, such as “comprise”, “include”, or ‘have”, are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

Now, an embodiment of the present disclosure will be explained in detail with reference to the accompanying drawings.

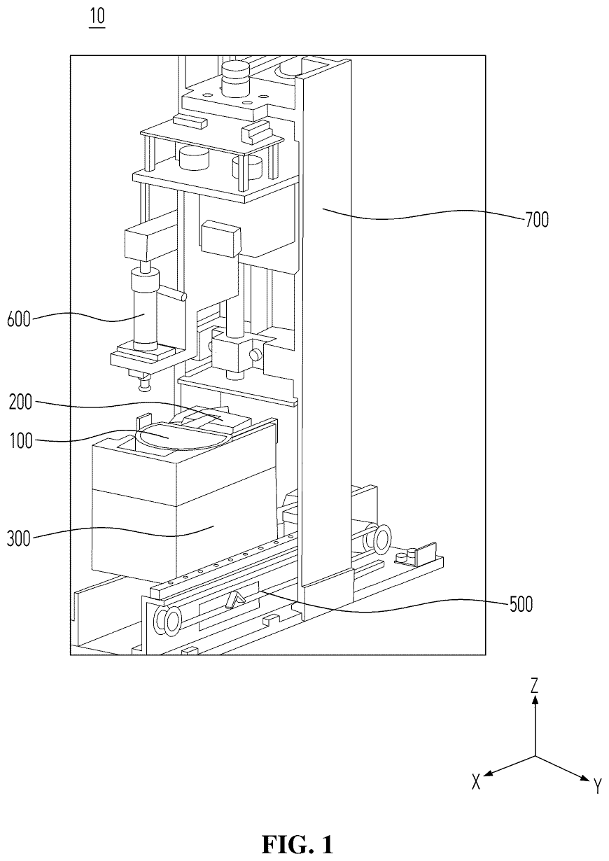

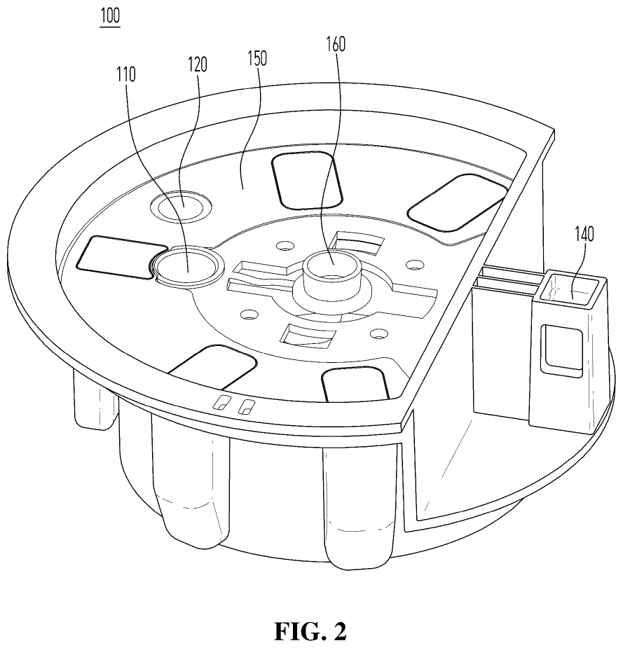

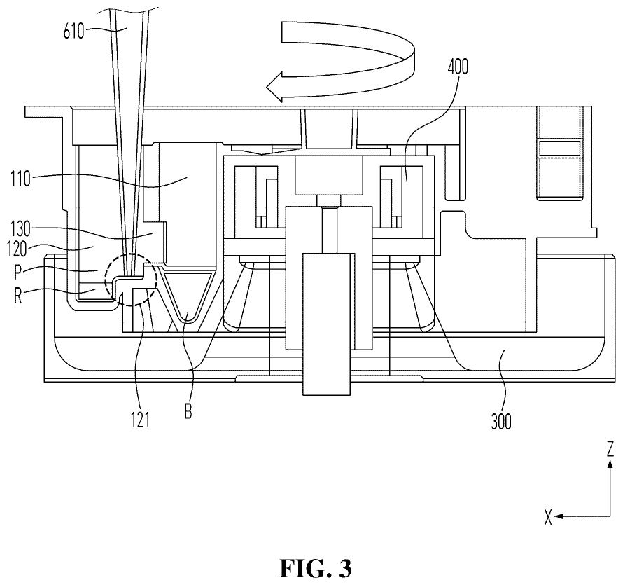

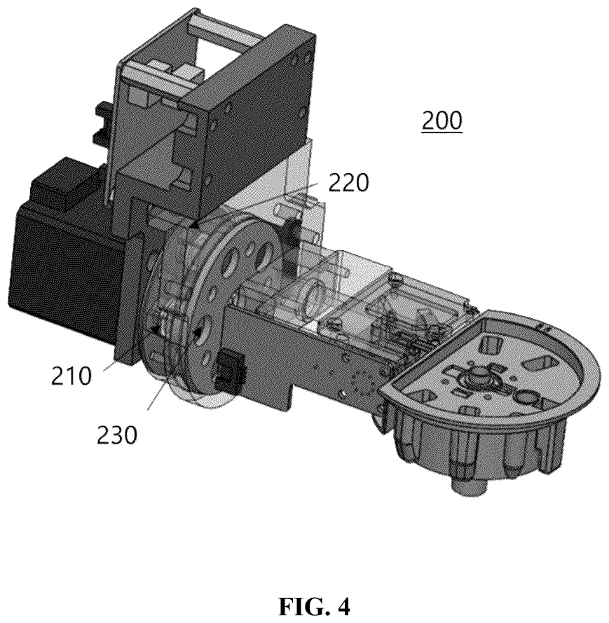

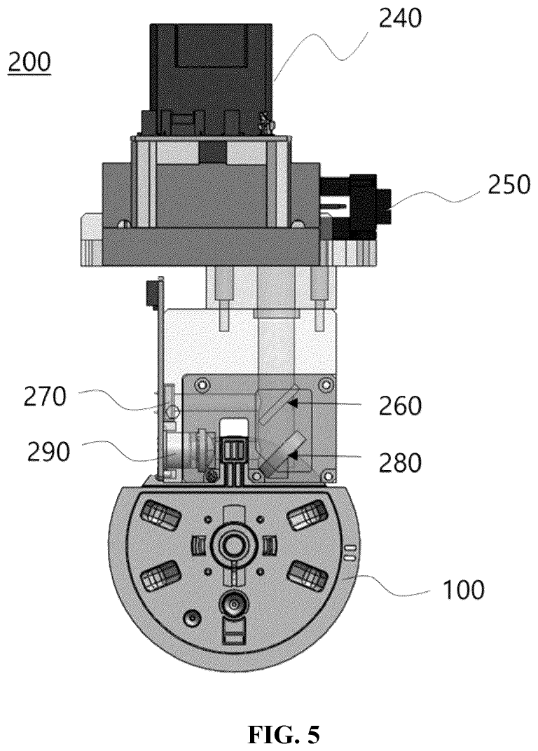

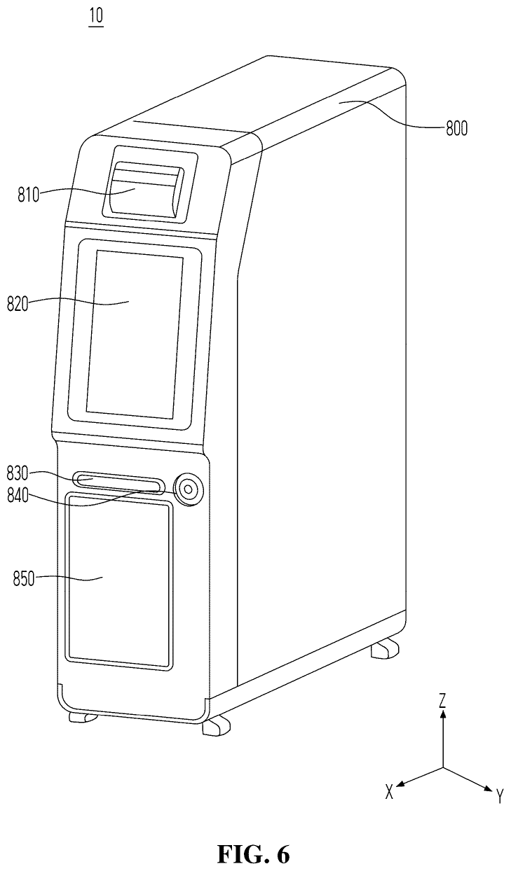

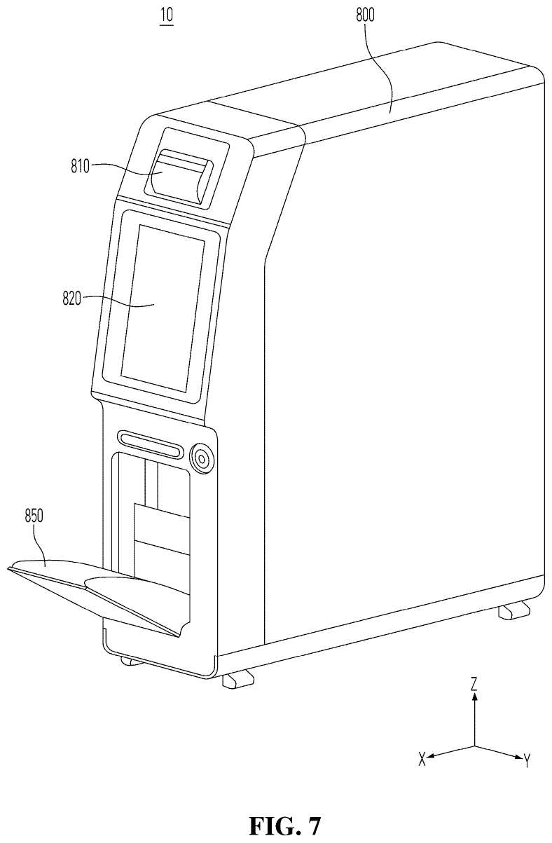

FIG. 1 is a perspective view showing a centrifugal immunoassay device according to the present disclosure, in which a housing is removed, FIG. 2 is a perspective view showing a centrifugal cartridge of the centrifugal immunoassay device according to the present disclosure, FIG. 3 is a sectional view showing the centrifugal cartridge according to the present disclosure, FIG. 4 is a perspective view showing an absorbance optical system of the centrifugal immunoassay device according to the present disclosure, FIG. 5 is a schematic view showing the absorbance optical system of the centrifugal immunoassay device according to the present disclosure, and FIGS. 6 and 7 are perspective views showing the centrifugal immunoassay device according to the present disclosure.

Referring to FIGS. 1 to 7, a centrifugal immunoassay device 10 according to an embodiment of the present disclosure is a device that separates an object to be detected from a sample and detects the separated object. In this case, the centrifugal immunoassay device 10 includes a centrifugal cartridge 100, an absorbance optical system 200, a cartridge-loading part 300, a rotating part 400, a horizontal driving part 500, a syringe 600, a syringe-driving part 700, a housing 800, and a controller.

The centrifugal cartridge 100 is an all-in-one cartridge that is mounted on the centrifugal immunoassay device 10 and rotates to separate the object to be detected from the sample stored therein and then detect the separated object. In this case, the centrifugal cartridge 100 includes a sample well 110, a centrifugal well 120, a sample-moving path 130, a detection well 140, a cover 150, and a tube cap 160.

The sample well 110 is a space into which the sample is injected by a user and stored before centrifugation is performed. In this case, the sample may be whole blood B. Otherwise, the sample may be a urine sample.

The centrifugal well 120 is a space to which the sample stored in the sample well 110 moves through the rotations and is then received in a state of being separated. That is, the centrifugal well 120 is spaced apart from the sample well 110 and thus receives the sample moving from the sample well 110 by means of the centrifugal force generated from the rotations of the centrifugal cartridge 100. Further, the centrifugal well 120 may be a space in which a sample centrifuged according to specific gravities of the substances constituting the sample is received.

In this case, the sample received in the sample well 110 moves to the centrifugal well 120 via the sample-moving path 130. The sample-moving path 130 is a path that is formed by connecting the sample well 110 and the centrifugal well 120 to each other and moves the sample only in a direction from the sample well 110 to the centrifugal well 120. In this case, the sample-moving path 130 is formed at a position having a given height or above to prevent the centrifuged sample from moving back to the sample well 110. In this case, the given height is a height of the sample received in the sample well 110 before the centrifugation is performed.

A substance having a high specific gravity in the centrifuged sample settles down at the bottom of the centrifugal well 120, and a substance having a low specific gravity in the centrifuged sample is located on a top of the substance having the high specific gravity so that the substance is separated from the settled substance. According to an embodiment of the present disclosure, the substance having the low specific gravity in the centrifuged sample is the object to be detected. In this case, the object to be detected is plasma P. According to another embodiment of the present disclosure, the substance having the high specific gravity in the centrifuged sample is the sample, not the object to be detected. In this case, the sample, which is not the object to be detected, is erythrocyte R.

The centrifugal well 120 has a stepped portion 121 formed on a lower portion thereof to suck only the object to be detected. That is, the centrifugal well 120 has the stepped portion 121 formed on the lower portion thereof to suck only the object to be detected from the centrifuged sample.

The stepped portion 121 is a space from which the object to be detected received in the centrifugal well 120 is sucked by a tip member 610 as will be discussed later. The stepped portion 121 is a space in which the tip member 610 is inserted into an upper portion thereof to suck the object to be detected. The stepped portion 121 is formed at a given position where only the object to be detected as the substance having the low specific gravity in the centrifuged sample is located on the upper portion thereof. In this case, the given position is higher than a height of the settled substance having the high specific gravity.

As the centrifugal well 120 has the stepped portion 121, only the object to be detected is sucked by the tip member 610, thereby ensuring reproducibility and improving the accuracy of detection. Reproducibility refers to the consistency of measurements when the same objects are measured by using the same methods under a condition wherein at least one of a measurer, a device, a measuring location, and a measuring time is different.

The detection well 140 is a space in which the object to be detected received in the centrifugal well 120 is received for the detection of the object. That is, the detection well 140 is a space in which the object to be detected received in the centrifugal well 120, which is sucked by the tip member 610, is discharged from the tip member 610 and thus received.

The detection well 140 has the shape of a cuvette capable of allowing transmission type detection. The cuvette is a small tube-like container designed to store a sample in spectroscopy. The detection well 140 is made of a transparent material capable of transmitting the light generated from the absorbance optical system 200 as will be discussed later over the object to be detected received in the detection well 140 to thus measure the absorbance of the object.

According to an embodiment of the present disclosure, the detection well 140 is a plastic cuvette. The plastic cuvette is used for high-speed spectroscopy in which a measuring speed is more important than accuracy, and after used, the plastic cuvette is thrown away, thereby preventing contamination occurring when used again. Further, the plastic cuvette is made of polymethyl methacrylate (PMMA) and polystyrene (PS), which is made at a lower cost than the conventional cartridge. However, the present disclosure is not limited to the above embodiment, and According to another embodiment of the present disclosure, therefore, the detection well 140 is a glass cuvette. According to yet another embodiment of the present disclosure, the detection well 140 is a quartz cuvette. The quartz cuvette has better durability than the plastic or glass cuvette and passes light with longer wavelengths than the plastic or glass cuvette therethrough.

The cover 150 serves to seal a top of the centrifugal cartridge 100. That is, the cover 150 seals the top of the centrifugal cartridge 100 to prevent the sample stored in the centrifugal cartridge 100 from escaping from the centrifugal cartridge 100, while the centrifugation is being performed. In this case, the cover 150 seals the entire top of the centrifugal cartridge 100 except the sample well 110 into which the sample is injected, before the centrifugation is performed.

The cover 150 is made of a material through which the tip member 610 passes. According to an embodiment of the present disclosure, the cover 150 is an aluminum cover provided by means of thermal fusion. However, the present disclosure may not be limited thereto, and therefore, the cover 150 may be made of various materials through which the tip member 610 can pass.

The tube cap 160 serves to open and close the sample well 110. That is, the tube cap 160 is located on the top of the centrifugal cartridge 100 to open and close the sample well 110, thereby controlling the movement of the substance going in and out of the sample well 110. According to an embodiment of the present disclosure, the tube cap 160 is separated from the sample well 110 before the centrifugation so that the sample is injected into the sample well 110. According to another embodiment of the present disclosure, the tube cap 160 is coupled to the sample well 110, while the centrifugation is being performed, so that the sample is prevented from escaping from the sample well 110.

The absorbance optical system 200 is coupled to the detection well 140 in which the object to be detected is received and thus performs transmission type detection for the object received in the detection well 140. The absorbance optical system 200 serves to pass light through the detection well 140 having the shape of the cuvette to measure the absorbance of the object. According to an embodiment of the present disclosure, the absorbance optical system 200 includes a plurality of LED lenses 210, a plurality of color LEDs 220, and a plurality of bandpass filters 230, which have different wavelengths. The detection light emitted from the color LEDs 220 passes through the LED lenses 210 and the bandpass filters 230, sequentially and thus has given wavelengths. Like this, one LED lens 210, one color LED 220, and one bandpass filter 230 are combined to provide a light emission part that emits the detection light having a single wavelength. According to an embodiment of the present disclosure, a plurality of light emission parts that emit the detection light having different wavelengths are arranged in the form of a circle. As a result, detection is performed using the detection light emitted from any one of the plurality of light emission parts through a light selection part having a driving motor 240 and a position sensor 250.

As the detection is performed using the detection light having the given wavelength emitted from any one of the plurality of light emission parts, a diagnosis for one component is made, and if the detection is performed using detection light having a different wavelength from the above detection light through the light selection part, a diagnosis for another component is made.

A dichroic mirror (DM) filter 260 is a filter through which only the light having such a wavelength capable of allowing the object to be detected to absorb light to the maximum passes. That is, the DM filter 260 is a special filter designed to allow light having excitation wavelengths to be reflected and allow light having emission wavelengths to pass therethrough. Further, the absorbance optical system 200 has a monitoring photodiode (PD) 270 adapted to monitor the wavelength value of the light reflected by the DM filter 260 and constantly control the luminous intensity of the light emitted from the light emission part.

The light passing through the DM filter 260 is reflected onto a mirror 280 and passes through the detection well 140 in which the object to be detected is received. A detection value of the light passing through the detection well 140 is calculated by a light detection part having a detection PD 290.

The cartridge-loading part 300 receives the centrifugal cartridge 100 and the absorbance optical system 200 therein. That is, the cartridge-loading part 300 has a cartridge hole larger than the centrifugal cartridge 100 so that the centrifugal cartridge 100 can rotate in the cartridge hole. Further, the cartridge-loading part 300 is a space in which the centrifugal cartridge 100 completed in centrifugating the sample for the measurement of the absorbance of the object is coupled to the absorbance optical system 200.

The rotating part 400 is located inside the cartridge-loading part 300 and rotates therein, so that on top of the rotating part 400 is mounted the centrifugal cartridge 100, thereby allowing the centrifugation to be performed. According to an embodiment of the present disclosure, the rotating part 400 rotates at a high speed of 4000 rpm. The rotating part 400 performs the high-speed rotation to move the sample stored in the sample well 110 to the centrifugal well 120 via the sample-moving path 130 and separate the sample moving to the centrifugal well 120 into the object to be detected and the sample that is not the object to be detected.

According to another embodiment of the present disclosure, the rotating part 400 performs step rotations. That is, the rotating part 400 performs the step rotations to allow a central portion of the tip member 610 and a central portion of the centrifugal well 120 to be placed on the same line as each other when the tip member 610 is inserted into the centrifugal well 120 to suck the object to be detected. Further, the rotating part 400 performs the step rotations to allow the central portion of the tip member 610 and a central portion of the detection well 140 to be placed on the same line as each other when the tip member 610 sucking the object to be detected discharges the object to the detection well 140.

The horizontal driving part 500 is located on the underside of the cartridge-loading part 300 to move the cartridge-loading part 300 horizontally. That is, the horizontal driving part 500 is located on any one of both sides of the cartridge-loading part 300, when the cartridge-loading part 300 is viewed on a front side (e.g., in a direction parallel to a ZY-plane) thereof, to move the cartridge-loading part 300 in a horizontal direction (e.g., in a direction of an X or −X-axis). In this case, the horizontal driving part 500 has a guide rail adapted to move the cartridge-loading part 300 in the horizontal direction.

The guide rail extends to be parallel to the moving direction (the direction of the X-axis) of the horizontal driving part 500. In this case, the cartridge-loading part 300 has a guide protrusion protruding from a side underside thereof (in a direction of a Y-axis). According to an embodiment of the present disclosure, the guide protrusion extends from the side underside edge of the cartridge-loading part 300 in such a way as to be connected to the guide rail.

According to another embodiment of the present disclosure, the guide protrusion extends from the side underside edge of the cartridge-loading part 300, while being formed on a portion of the side underside edge of the cartridge-loading part 300. In this case, the guide rail extends to be parallel to the guide protrusion so that one surface (hereinafter, referred to as a guide surface) of the guide rail is placed to face one surface of the guide protrusion. If the cartridge-loading part 300 moves in the horizontal direction (e.g., in the direction of the X or −X-axis), as a result, the guide protrusion slides along the guide surface of the guide rail to prevent the cartridge-loading part 300 from tilting.

The syringe 600 is located above the cartridge-loading part 300 and serves to suck the object to be detected and inject it into the detection well 140. That is, the syringe 600 is inserted into the upper portion of the stepped portion 121 formed on the lower portion of the centrifugal well 120 to suck the object to be detected, and then, the syringe 600 discharges the sucked object to the detection well 140. In this case, the syringe 600 is provided with the tip member 610. The tip member 610 is located on the bottom of the syringe 600 to suck and discharge the object to be discharged. The tip member 610 passes through the top cover 150 of the centrifugal cartridge 100 and sucks the object received in the centrifugal well 120. In this case, the tip member 610 becomes small in diameter as it goes toward the end portion thereof so that it passes through the top cover 150. After that, the tip member 610 discharges the sucked object to the detection well 140 in which the absorbance of the object is measured.

The syringe-driving part 700 moves the syringe 600. That is, the syringe-driving part 700 is driven in upward and downward directions (e.g., in directions of Z and −Z-axes) to allow the syringe 600 to suck and discharge the object to be detected. According to an embodiment of the present disclosure, the syringe-driving part 700 is driven in the downward direction (e.g., in the direction of the −Z-axis) to allow the syringe 600 to suck the object received in the centrifugal well 120. According to another embodiment of the present disclosure, the syringe-driving part 700 is driven in the upward direction (e.g., in the direction of the Z-axis) to allow the syringe 600 to discharge the sucked object to the detection well 140.

The housing 800 serves to surround the centrifugal cartridge 100, the absorbance optical system 200, the cartridge-loading part 300, the rotating part 400, the horizontal driving part 500, the syringe 600, and the syringe-driving part 700. In this case, the housing 800 includes a printer 810, a touch screen 820, a progress indicator 830, a power switch 840, and a door 850.

The printer 810 is a thermal printer that applies heat to thermal paper to transfer characters or pictures on the paper. That is, the printer 810 is a thermal printer as a non-impact printer that heats a recording head to apply colors to thermal paper so that characters or pictures are printed in the form of dots. However, the present disclosure may not be limited thereto. In this case, the printer 810 is located on an upper end of the housing 800 when the housing 800 is viewed on the front side (e.g., in the direction parallel to the ZY-plane) thereof.

The touch screen 820 receives the commands for performing the operations of the centrifugal immunoassay device 10 from the user. The touch screen 820 is a contact type display for receiving the commands from the user's body such as a finger, a palm of the hand, and the like and from a means for touch.

The touch screen 820 is provided with command-receiving touch buttons allowing a plurality of operations of the centrifugal immunoassay device 10 to be performed. According to an embodiment of the present disclosure, the touch screen 820 has a start button and a pause button. However, the present disclosure may not be limited thereto. According to another embodiment of the present disclosure, the touch screen 820 has a rotation operation button for performing the centrifugation through the rotating part 400 and a detection operation button for moving the centrifuged object to the detection well 140 and performing the detection for the object.

The progress indicator 830 is located on the front surface of the housing 800 (e.g., in the direction parallel to the ZY-plane) and indicates a progress in the operation of the centrifugal immunoassay device 10 to the outside so that the progress is checked by the user.

The power switch 840 is a switch that turns on or off the power of the centrifugal immunoassay device 10. In this case, the power switch 840 is located on the front surface of the housing 800 (e.g., in the direction parallel to the ZY-plane).

The door 850 is open and closed to allow the centrifugal cartridge 100 to be mounted on the centrifugal immunoassay device 10 by the user. In this case, the door 850 is located on the front surface of the housing 800 (e.g., in the direction parallel to the ZY-plane).

The controller controls the operations of the centrifugal immunoassay device 10, based on the commands received through the touch screen 820 from the user. That is, the controller controls the absorbance optical system 200, the rotating part 400, the horizontal driving part 500, the syringe 600, and the syringe-driving part 700, based on the commands received through the touch screen 820 from the user.

According to an embodiment of the present disclosure, if the start button of the touch screen 820 is pressed, the controller controls the absorbance optical system 200, the rotating part 400, the horizontal driving part 500, the syringe 600, and the syringe-driving part 700 to perform the centrifugation of the sample through the rotation of the rotating part 400, the upward or downward movement of the syringe 600 by means of the syringe-driving part 700, the object-sucking and discharging operations of the tip member 610 of the syringe 600, and the object-detecting operation through the coupling between the centrifugal cartridge 100 and the absorbance optical system 200.

If a trouble occurs while the centrifugal immunoassay device 10 is operating, further, the pause button of the touch screen 820 is pressed to allow the corresponding operation of the centrifugal immunoassay device 10 to be paused.

As described above, the centrifugal immunoassay device according to the embodiments of the present disclosure can perform the centrifugation and transmission type detection through one centrifugal cartridge, thereby completing a detection process in the single device. Further, the centrifugal immunoassay device according to the embodiments of the present disclosure can perform the centrifugation and detection through the single centrifugal immunoassay device and the single centrifugal cartridge and then check the detected result, thereby efficiently reducing a detection time and cost.

Furthermore, the centrifugal immunoassay device according to the embodiments of the present disclosure can measure the plurality of wavelengths so that it performs multiple diagnoses, thereby reducing a detection time and cost.

The foregoing description of the embodiments of the disclosure has been presented for the purpose of illustration; it is not intended to be exhaustive or to limit the disclosure to the precise forms disclosed. Persons skilled in the relevant art can appreciate that many modifications and variations are possible in light of the above teachings. For example, the parts expressed in a singular form may be dispersedly provided, and in the same manner as above, the parts dispersed may be combined with each other.

Accordingly, the present subject matter is not limited to the embodiments described above or depicted in the drawings, and various embodiments and modifications can be made without departing from the scope of the claims below.

What is claimed is:

1. A centrifugal immunoassay device for multiple diagnoses for detecting an object to be detected from a sample through centrifugation, comprising:
a centrifugal cartridge having a plurality of wells in which the sample is received;
a rotating part rotating in a state of mounting the centrifugal cartridge thereon;
an absorbance optical system coupled to the centrifugal cartridge to measure the absorbance of the object to be detected received in a detection well among the plurality of wells of the centrifugal cartridge;
a syringe adapted to be inserted into the centrifugal cartridge, suck the object to be detected, and discharge the sucked object to the detection well; and
a housing for surrounding the centrifugal cartridge, the rotating part, the absorbance optical system, and the syringe,
wherein the absorbance optical system comprises:
a plurality of light emission parts for emitting light having given wavelengths in different ranges to the object to be detected received in the detection well;
a light detection part for measuring the light passing through the object to be detected received in the detection well to calculate a detection value of the light; and
a light selection part for selecting the light having the given wavelength in any one range emitted from one of the plurality of light emission parts to transmit the selected light to the objected to be detected received in the detection well.

2. The centrifugal immunoassay device according to claim 1, wherein the sample is blood and the object to be detected is plasma.

3. The centrifugal immunoassay device according to claim 1, wherein the detection well has the shape of a cuvette capable of allowing transmission type detection.

4. The centrifugal immunoassay device according to claim 1, wherein the centrifugal cartridge comprises:
a sample well for receiving the sample therein;
a centrifugal well for receiving the sample moving thereto from the sample well through rotations;
a sample-moving path formed by connecting the sample well and the centrifugal well to each other; and
the detection well for receiving the object to be detected from the syringe.

5. The centrifugal immunoassay device according to claim 4, wherein the syringe has a tip member located on the bottom thereof and the tip member passes through a top of the centrifugal cartridge, sucks the object to be detected received in the centrifugal well, and then discharges the sucked object to be detected to the detection well.

6. The centrifugal immunoassay device according to claim 5, wherein the centrifugal well has a stepped portion formed on a lower portion thereof to suck the object to be detected separated from the sample and the tip member is inserted into an upper portion of the stepped portion to suck the object to be detected.

7. The centrifugal immunoassay device according to claim 1, wherein the plurality of light emission parts are arranged in the form of a circle, and the light selection part comprises a driving motor for rotating the plurality of light emission parts arranged in the form of the circle and a position sensor for detecting the positions of the plurality of light emission parts.

8. The centrifugal immunoassay device according to claim 1, wherein each light emission part comprises:
a color LED for emitting the light having the given wavelength;
an LED lens for focusing the light emitted from the color LED; and
a bandpass filter for passing the light having the given wavelength therethrough.

9. The centrifugal immunoassay device according to claim 1, wherein the housing comprises at least any one of a printer, a touch screen, a progress indicator, a power switch, and a door.

10. The centrifugal immunoassay device according to claim 9, wherein the touch screen receives commands for performing operations of the centrifugal immunoassay device for multiple diagnoses from a user.
